(19) United States
(12) Patent Application Publication
(10) Pub. No.: US 2026/0199651 A1
(43) Pub. Date: Jul. 16, 2026

(54) DOSING DEVICE AND INFORMATION ACQUISITION SYSTEM FOR DOSING DEVICE

(71) Applicant: Murata Manufacturing Co., Ltd., Nagaokakyo-shi (JP)

(72) Inventor: Yuta NAKAHIGASHI, Nagaokakyo-shi (JP)

(21) Appl. No.: 19/564,366

(22) Filed: Mar. 12, 2026

Related U.S. Application Data
(63) Continuation-in-part of application No. PCT/JP2024/032140, filed on Sep. 9, 2024.

(30) Foreign Application Priority Data
Sep. 14, 2023 (JP) 2023-149436

(51) Int. Cl. A61M 31/00; A61H 23/02

(52) U.S. Cl. CPC A61M 31/002; A61H 23/0245

(57) ABSTRACT

A dosing device of the present disclosure is a dosing device that emits information by vibration of the device itself, the device including an ultrasonic oscillator inside, in which a curvature of an outer surface located on a vibration direction of the ultrasonic oscillator is 0.18 or more and 0.50 or less.

### CROSS REFERENCE TO RELATED APPLICATIONS

This application is a continuation-in-part of International Application No. PCT/JP2024/032140, filed Sep. 9, 2024, which claims priority to Japanese Patent Application No. 2023-149436, filed Sep. 14, 2023, the contents of each of which are hereby incorporated by reference in their entireties.

### TECHNICAL FIELD

The present disclosure relates to a dosing device and an information acquisition system for the dosing device.

### BACKGROUND

There is a need for a method of checking whether a patient has taken a prescribed medicine and a method of checking what biological reaction may occur by a patient taking a medicine. In this regard, development of a medication device that transmits a signal from inside a body to the outside of the body after being taken with a medicine is in progress.

Japanese Patent No. 6914567 (the “'567 patent”) describes a swallowing sensor device including sensors including a sensor and a device for wirelessly transmitting information detected by the sensor, and a substrate group configured by stacking a plurality of rigid substrates.

However, since the swallowing sensor device described in the '567 patent performs communication in a radio wave system, there is a possibility that a signal is emitted non-directionally regardless of the inside and outside of the body and is acquired by a third party, and there is a problem in terms of security.

On the other hand, it is conceivable that a dosing device that emits an ultrasonic signal is used, and the ultrasonic signal emitted from the dosed dosing device is received by, for example, a receiver attached to the body surface of the recipient. According to this method, the ultrasonic signal emitted from the dosing device basically propagates in the body of the recipient, but is reflected at the interface between the body and the air, and thus hardly leaks into the air, so that the possibility of being acquired by the third party can be reduced.

However, since the ultrasonic signal is easily attenuated by the obstacle, there is room for contrivance in terms of how to improve the sound pressure of the ultrasonic signal emitted from the dosing device.

### SUMMARY OF THE INVENTION

Accordingly, it is an object of the present disclosure to provide a dosing device that improves the sound pressure of an ultrasonic signal to be emitted. Moreover, an information acquisition system for the dosing device that includes the dosing device is also provided.

A dosing device of the present disclosure is a dosing device that emits information by vibration of the device itself, the device including an ultrasonic oscillator inside, in which a curvature of an outer surface located on a vibration direction of the ultrasonic oscillator is 0.18 or more and 0.50 or less.

An information acquisition system for a dosing device of the present disclosure includes the dosing device of the present disclosure, a processing device, and a receiver that receives an oscillating ultrasonic signal from the ultrasonic oscillator to acquire information from the dosing device and transmits the acquired information to the processing device.

According to the present disclosure, it is possible to provide a dosing device for improving the sound pressure of an ultrasonic signal to be emitted, and an information acquisition system for the dosing device including the dosing device.

### BRIEF DESCRIPTION OF DRAWINGS

In the descriptions that follow, like parts are marked throughout the specification and drawings with the same numerals, respectively. The drawings are not necessarily drawn to scale and certain drawings may be illustrated in exaggerated or generalized form in the interest of clarity and conciseness. The disclosure itself, however, as well as a mode of use, further features and advances thereof, will be understood by reference to the following detailed description of illustrative implementations of the disclosure when read in conjunction with reference to the accompanying drawings, wherein:

FIG. 1 is a perspective view schematically illustrating an example of a dosing device in accordance with aspects of the present disclosure;

FIG. 2 is a diagram schematically illustrating an example of a cross section along a line segment A1-A1 of the dosing device illustrated in FIG. 1 in accordance with aspects of the present disclosure;

FIG. 3 is a diagram schematically illustrating an example of a cross section along a line segment A2-A2 of the dosing device illustrated in FIGS. 1 and 2 in accordance with aspects of the present disclosure;

FIG. 4 is a graph illustrating a relationship between the sound pressure ratio and the curvature of the outer surface located on the vibration direction of the ultrasonic oscillator, obtained from the simulation in accordance with aspects of the present disclosure;

FIG. 5A is a sectional view schematically illustrating another example of the dosing device in accordance with aspects of the present disclosure;

FIGS. 5B to 5F are sectional views schematically illustrating additional exemplary aspects of the dosing device that are a variation of the example shown in FIG. 5A and are in further accordance with aspects of the present disclosure;

FIG. 6 is a plan view schematically illustrating an example of an arrangement relationship between a battery and a substrate on which the battery is mounted in the dosing device in accordance with aspects of the present disclosure;

FIG. 7 is a diagram schematically illustrating an analysis model used to simulate a maximum sound pressure of an ultrasonic signal emitted from the dosing device in accordance with aspects of the present disclosure;

FIG. 8 is a graph showing a relationship between a maximum sound pressure of an ultrasonic signal and a thickness of a sealant, obtained from a simulation using the analysis model illustrated in FIG. 7 in accordance with aspects of the present disclosure; and

FIG. 9 is a block diagram schematically illustrating an example of an information acquisition system of a dosing device in accordance with aspects of the present disclosure.

### DETAILED DESCRIPTION

Hereinbelow, aspects of the present disclosure will be described. In a following description of the drawings, the same or similar components will be represented with use of the same or similar reference characters. The drawings are exemplary, sizes or shapes of portions are schematic, and technical scope of the present disclosure should not be understood with limitation to the aspects.

Hereinafter, a dosing device and an information acquisition system for the dosing device of the present disclosure will be described. The present disclosure is not limited to the following configuration, and may be modified as appropriate without changing the gist of the present disclosure. The present disclosure also includes a combination of a plurality of individual preferable configurations described below.

It goes without saying that each of the aspects illustrated below is an example, and partial replacement or combination of configurations illustrated in different aspects is possible. In one aspect and subsequent aspects, matters common to one aspect will not be described, and only a different point will be mainly described. In particular, the same operation and effect by the same configuration will not be sequentially mentioned for each aspect.

In the following description, when the aspects are not particularly distinguished, the dosing device of each aspect is simply referred to as the “dosing device according to the present disclosure”.

In the present specification, the terms indicating the relationship between elements (for example, “vertical”, “parallel”, and “orthogonal”) and the terms indicating the shape of an element are not expressions indicating only a strict meaning, but are expressions meaning to include a substantially equivalent range, for example, a difference of about several %.

The following drawings are schematic views, and the dimensions, the scales of aspect ratios, and the like may be different from those of actual products.

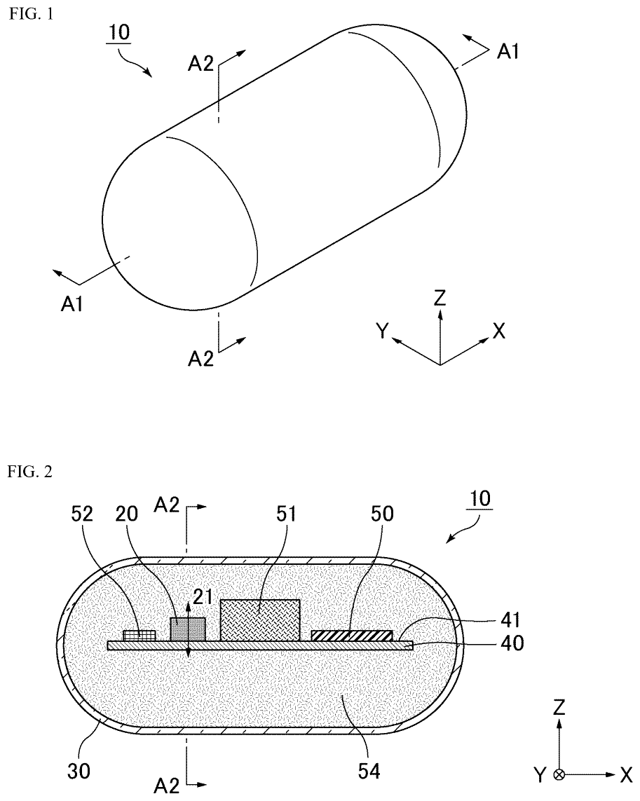

FIG. 1 is a perspective view schematically illustrating an example of a dosing device in accordance with an aspect of the present disclosure. FIG. 2 is a diagram schematically illustrating an example of a cross section along a line segment A1-A1 of the dosing device illustrated in FIG. 1. In each drawing, the X-axis direction is a longitudinal direction of the dosing device, and the Z-axis direction is a lateral direction of the dosing device. The Y axis is an axis orthogonal to each of the X axis and the Z axis.

A dosing device 10 illustrated in FIG. 1 can emit information by the device 10 itself vibrating. The dosing device 10 is taken by a person with or without a medicine. The dosing device 10 itself may be taken alone. The information emitted from the dosing device 10 is not particularly limited, and may be a signal. Specifically, for example, a signal indicating that the medicine is taken or medicated together with the dosing device 10, biological information acquired in the body, and the like can be cited. The medicine to be taken with the dosing device 10 may be any medicine. Specifically, for example, the medicine may be a medicine to be taken by a person who has difficulty in recognizing the ingestion act itself. In this case, the medicine intake rate of such people can be improved. In addition, the burden on those who care for such people can also be reduced. Note that, in the present specification, “emitting” is synonymous with “transmitting” unless otherwise specified.

As illustrated in FIG. 2, the dosing device 10 includes an ultrasonic oscillator 20 therein, and drives the ultrasonic oscillator 20 to cause an ultrasonic signal output from the ultrasonic oscillator 20 to oscillate. The oscillating ultrasonic signal from the ultrasonic oscillator 20 propagates through the medium inside the dosing device 10 and is finally emitted to the outside of the dosing device 10. That is, the dosing device 10 itself vibrates by the oscillating ultrasonic signal from the ultrasonic oscillator 20, and emits information as an ultrasonic signal to the outside of the dosing device 10, specifically, to the body.

The ultrasonic oscillator 20 oscillates an ultrasonic wave, and the oscillating ultrasonic wave from the ultrasonic oscillator 20 has directivity in a direction in which a vibration surface of the ultrasonic oscillator 20 vibrates. The oscillation principle of an ultrasonic wave by the ultrasonic oscillator 20 is not particularly limited, but the ultrasonic oscillator 20 preferably includes a piezoelectric element, and preferably causes an ultrasonic wave to oscillate by vibrating the piezoelectric element. The piezoelectric element included in the ultrasonic oscillator 20 is also called a piezoelectric vibrator. Materials that can be used for the piezoelectric element include lead zirconate titanate (PZT), barium titanate (BT), and potassium sodium niobate (KNN). In that case, the dosing device 10 includes an IC, and the piezoelectric element is controlled by the IC. The oscillating ultrasonic signal from the ultrasonic oscillator 20 is received by a receiver attached to a body surface such as a torso, a neck, and a wrist of a recipient with a fixture such as a belt. The receiver is mounted to be in direct contact with the body surface or to be in contact with the body surface via an inclusion such as a gel.

The shapes of the ultrasonic oscillator 20 and the piezoelectric element are not particularly limited, and examples thereof include a cubic shape, a rectangular parallelepiped shape, and a disk shape.

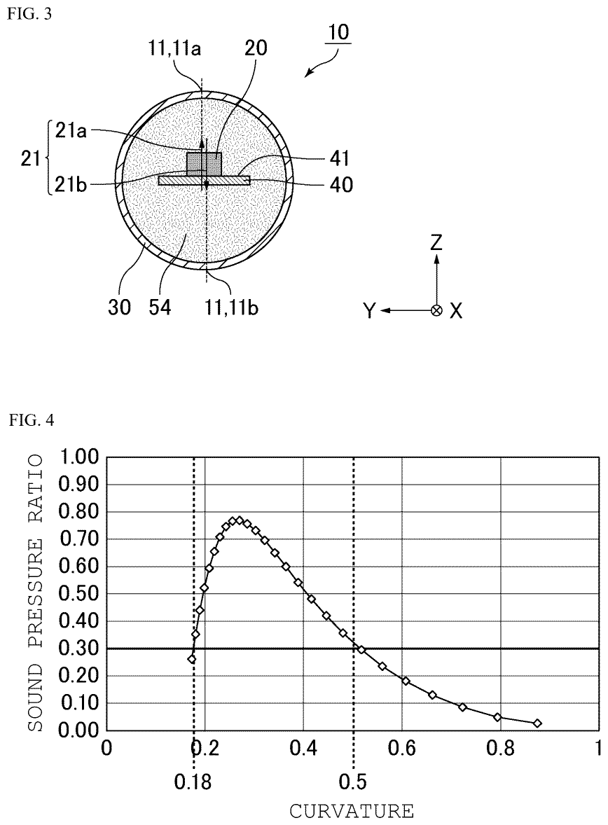

FIG. 3 is a diagram schematically illustrating an example of a cross section along a line segment A2-A2 of the dosing device illustrated in FIGS. 1 and 2.

As illustrated in FIG. 3, the dosing device 10 has an outer surface 11 located on a vibration direction 21 of the ultrasonic oscillator 20, and the curvature of the outer surface 11 is 0.18 or more and 0.50 or less. As a result, the sound pressure of the ultrasonic signal emitted from the dosing device 10 can be remarkably improved.

More specifically, by setting the curvature of the outer surface 11 to 0.18 or more and 0.50 or less, the sound pressure ratio can be set to 0.3 or more. Here, the sound pressure ratio is a value calculated from the expression: Sound Pressure Ratio=(Sound Pressure at Position of Receiver)/(Sound Pressure immediately after Oscillation from Ultrasonic Oscillator). When the sound pressure ratio is 0.3 or more, it is possible to secure the sound pressure necessary for the ultrasonic signal emitted from the dosing device 10 in the body to propagate through the body and reach the receiver.

Here, the “vibration direction of the ultrasonic oscillator” means a direction in which a displacement amount at a frequency to be driven is the largest.

In the present specification, the “curvature of the outer surface located on the vibration direction of the ultrasonic oscillator” is calculated by the following method. That is, first, a sectional image that is a sectional image of the dosing device and includes the outer surface located on the vibration direction of the ultrasonic oscillator is acquired. Next, curve fitting of the outer surface portion is performed to obtain an ellipse obtained by extrapolating the outer surface portion, and a major axis a and a minor axis b of the ellipse and a parameter θ (0≤θ<2π) of a point located in the vibration direction when the ellipse is displayed as a parameter are obtained. Then, the obtained a, b, and θ are substituted into the following curvature k formula to calculate the curvature of the outer surface located on the vibration direction of the ultrasonic oscillator.

$$κ = \frac{ab}{{( {a}^{2} {sin}^{2} θ + {b}^{2} {cos}^{2} θ )}^{\frac{3}{2}}} \qquad Equation 1$$

Note that the curvature to be calculated can vary depending on how the sectional image including the outer surface located on the vibration direction of the ultrasonic oscillator is taken, that is, the direction of the sectional image. However, in the present specification, the largest curvature among the curvatures that can be calculated by the above method is referred to as “the curvature of the outer surface located on the vibration direction of the ultrasonic oscillator”.

As illustrated in FIG. 3, the vibration direction 21 of the ultrasonic oscillator 20 may include a first direction 21a and a second direction 21b diametrically opposite to the first direction 21a, in which case the outer surface 11 located on the vibration direction 21 of the ultrasonic oscillator 20 will include a first outer surface 11a located at a place straight from the ultrasonic oscillator 20 to the first direction 21a and a second outer surface 11b located at a place straight from the ultrasonic oscillator 20 to the second direction 21b. In this case, the curvature of at least one of the first outer surface 11a and the second outer surface 11b may be 0.18 or more and 0.50 or less, but the curvature of both the first outer surface 11a and the second outer surface 11b may be 0.18 or more and 0.50 or less.

The curvature of the outer surface 11 is preferably 0.20 or more and 0.40 or less. As a result, the sound pressure, for example, the sound pressure ratio of the ultrasonic signal emitted from the dosing device 10 can be particularly remarkably improved. Similarly, the curvature of the first outer surface 11a and/or the second outer surface 11b is preferably 0.20 or more and 0.40 or less.

Here, a result of simulating the sound pressure ratio of the ultrasonic signal by changing the curvature of the outer surface located on the vibration direction 21 of the ultrasonic oscillator 20 will be described.

FIG. 4 is a graph illustrating a relationship between the sound pressure ratio and the curvature of the outer surface located on the vibration direction of the ultrasonic oscillator, obtained from the simulation. As shown above, the numerator of the equation represents the sound pressure at the receiver position, which is the sound pressure immediately before reception. The denominator represents the sound pressure immediately after emission from the transmitter. Therefore, in the context of FIG. 4, the sound pressure ratio is the ratio of these values, making it dimensionless. For the Y-axis, the unit is [mm⁻¹]. Thus, as an example, a curvature of 0.5 mm⁻¹ indicates a curvature radius of 2 mm (e.g., the curvature of a circle with a diameter of 4 mm). Similarly, a curvature of 0.18 mm⁻¹ indicates a curvature radius of 5.56 mm (the curvature of a circle with a diameter of 11.1 mm).

As further illustrated in FIG. 4, when the curvature of the outer surface 11 located on the vibration direction 21 of the ultrasonic oscillator 20 is 0.18 or more and 0.50 or less, the sound pressure ratio can be 0.3 or more. In addition, when the curvature of the outer surface 11 located on the vibration direction 21 of the ultrasonic oscillator 20 is 0.20 or more and 0.40 or less, the sound pressure ratio can be 0.5 or more.

Referring back to FIG. 2, the dosing device 10 may include a capsule-shaped housing 30, a substrate 40, a power receiving coil 50, a battery 51, a biological information acquisition unit 52, an A/D converter (not illustrated), and a sealant 54.

The ultrasonic oscillator 20 is disposed inside the housing 30.

The housing 30 is made of, for example, a biocompatible resin or a general-purpose resin having a surface coated with a biocompatible material. For example, an epoxy resin can be used as a biocompatible resin. A material preferably used for the housing 30 is a material that is discharged to the outside of the body without being dissolved by gastric acid or the like after the dosing device 10 is taken into the body.

The outer surface 11 having the curvature may be an outer surface of the housing 30. That is, the first outer surface 11a and/or the second outer surface 11b may be an outer surface of the housing 30. In addition, substantially the entire outer surface of the dosing device 10 may be constituted by the outer surface of the housing 30.

Note that FIGS. 1 to 3 illustrate the cylindrical housing 30 having the so-called capsule shape, specifically hemispherical both ends in the longitudinal direction, but the outer shapes of the dosing device 10 and the housing 30 are not particularly limited as long as they do not interfere with dosage, and may be spherical, ellipsoidal, discoid, cylindrical, tablet-shaped, polygonal columnar with rounded corners, or the like. The ellipsoid may be a prolate spheroid or an oblate spheroid.

The substrate 40 is disposed inside the housing 30. One or a plurality of substrates 40 may be disposed inside the housing 30. As a material of the substrate 40, for example, a glass epoxy resin, FR-4, or the like can be used.

The substrate 40 is arranged in parallel with the longitudinal direction of the dosing device 10. That is, the substrate 40 is disposed so as to be orthogonal to the lateral direction of the dosing device 10. Note that the longitudinal direction of the dosing device 10 is synonymous with the longitudinal direction of the housing 30, and the lateral direction of the dosing device 10 is synonymous with the lateral direction of the housing 30.

As illustrated in FIG. 2, the vibration direction 21 of the ultrasonic oscillator 20 may be a direction perpendicular to a mounting surface 41 of the substrate 40 on which the ultrasonic oscillator 20 is mounted.

In this case, the outer surface 11 having the curvature is preferably located on the opposite side of the substrate 40 with respect to the ultrasonic oscillator 20. That is, the substrate 40, the ultrasonic oscillator 20, and the outer surface 11 having the curvature may be arranged in this order on a straight line parallel to the vibration direction 21 of the ultrasonic oscillator 20. As a result, the ultrasonic signal can more efficiently oscillate from the ultrasonic oscillator 20 on the side opposite to the substrate 40.

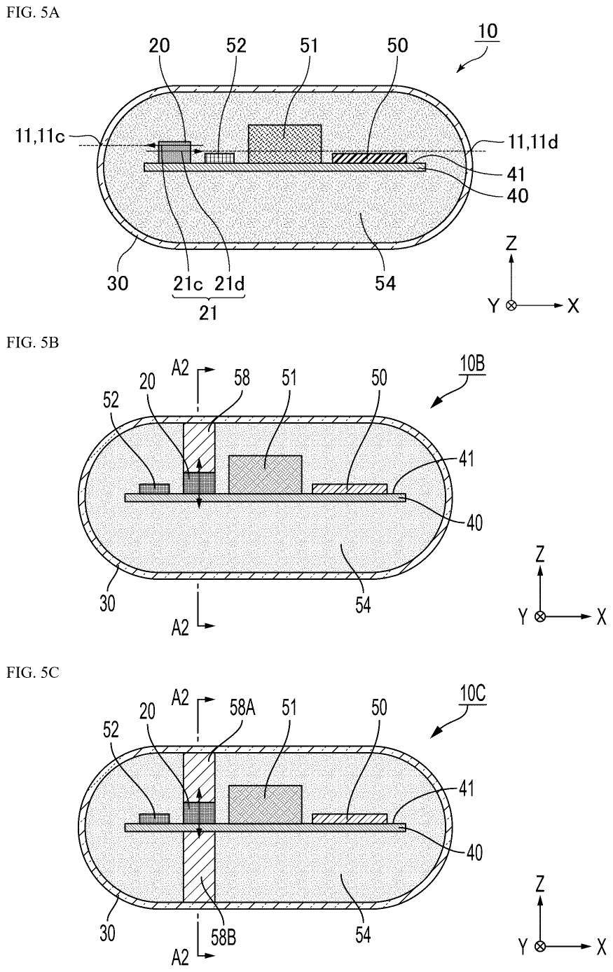

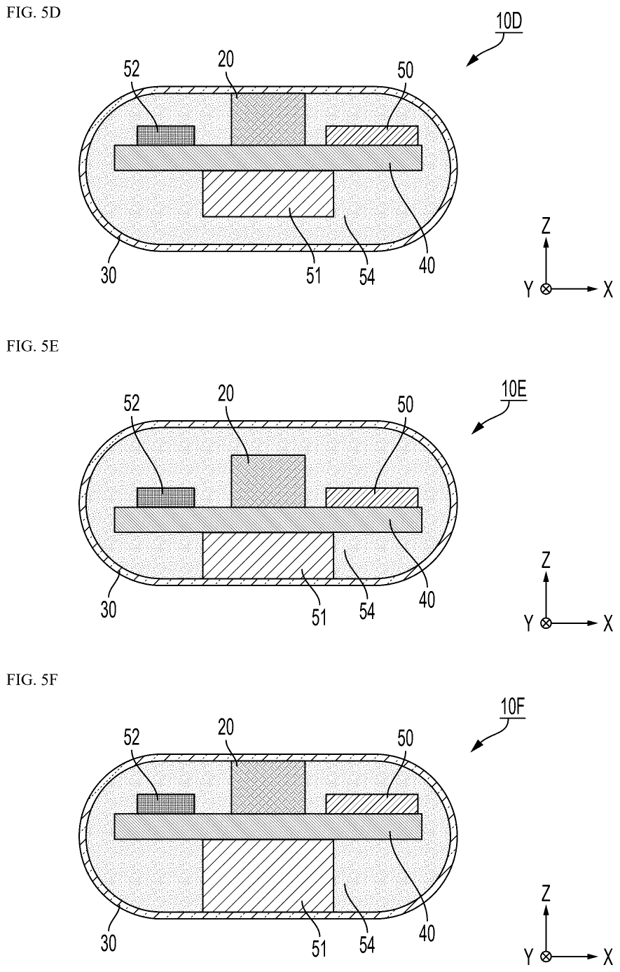

FIG. 5A is a sectional view schematically illustrating another example of the dosing device in accordance with another aspect of the present disclosure. FIG. 5A corresponds to the sectional view of FIG. 2.

As illustrated in FIG. 5A, the vibration direction 21 of the ultrasonic oscillator 20 may be a direction parallel to the mounting surface 41 of the substrate 40. Also in this case, the vibration direction 21 of the ultrasonic oscillator 20 may include a first direction 21c and a second direction 21d diametrically opposite to the first direction 21c, and the outer surface 11 located on the vibration direction 21 of the ultrasonic oscillator 20 may include a first outer surface 11c located at a place straight from the ultrasonic oscillator 20 in the first direction 21c and a second outer surface 11d located at a place straight from the ultrasonic oscillator 20 in the second direction 21d. The curvature of at least one of the first outer surface 11c and the second outer surface 11d may be 0.18 or more and 0.50 or less, and the curvature of each of the first outer surface 11c and the second outer surface 11d may be 0.18 or more and 0.50 or less. Also in this case, similarly to the case illustrated in FIG. 1, the curvature of the outer surface 11 is preferably 0.20 or more and 0.40 or less, and the curvature of the first outer surface 11c and/or the second outer surface 11d is preferably 0.20 or more and 0.40 or less.

The power receiving coil 50 is for performing wireless power supply in pair with the power transmission coil and is provided on the substrate 40. The power receiving coil 50 is made of, for example, copper. Wireless power supply may be performed using a technique using electromagnetic induction or magnetic field resonance. Magnetic field resonance causes a current to flow in the power receiving coil 50 by an effect of varying of the magnetic field generated by the power transmission coil, and is thus a type of electromagnetic induction. Although not illustrated in FIG. 2, the power receiving coil 50 is connected to the battery 51. The power receiving coil 50 is provided, for example, on the substrate 40 on which the battery 51 is provided. However, a place where the power receiving coil 50 is provided is not particularly limited to the substrate 40 on which the battery 51 is provided.

The battery 51 is, for example, a secondary battery is charged with the power received by the power receiving coil 50. In that case, the battery 51 is not particularly limited as long as it is a chargeable/dischargeable battery, and may be, for example, an all-solid-state battery having a solid electrolyte. The all-solid-state battery is suitable for the dosing device 10 because there is no liquid leakage. In addition, the battery 51 may be a primary battery as long as the current flows through the dosing device 10 immediately before dosing.

FIGS. 5B to 5F are sectional views schematically illustrating additional exemplary aspects of the dosing device that are a variation of the example shown in FIG. 5A and are in further accordance with aspects of the present disclosure.

In particular, FIG. 5B is a sectional view schematically illustrating yet another example of the dosing device in accordance with another aspect of the present disclosure. As shown, the dosing device 10B is substantially the same as the dosing device 10 illustrated in FIG. 5A, and includes the ultrasonic oscillator 20 including a piezoelectric element, the housing 30, the substrate 40, the power receiving coil 50, the battery 51, the biological information acquisition unit 52, and the sealant 54. In the aspect illustrated in FIG. 5B, the dosing device 10 further includes a vibration transmission unit 58 interposed between the ultrasonic oscillator 20 and an inner wall of the housing 30. According to this configuration, the piezoelectric element forming the ultrasonic oscillator 20 is connected to the inner wall of the housing 30 via the vibration transmission unit 58. The vibration transmission unit 58 provides a direct mechanical coupling between the piezoelectric element and the wall of the housing 30, so that vibration of the piezoelectric element propagates directly to the housing 30 via the vibration transmission unit 58. As a result, the ultrasonic waves are efficiently radiated from the dosing device 10, and the sound pressure of the ultrasonic signal emitted from the dosing device 10 can be further improved. Moreover, according to the exemplary aspect, the vibration transmission unit 58 is preferably made of a material having a higher acoustic impedance than the sealant 54. Examples of such materials that can be used for the vibration transmission unit 58 include epoxy resin, polyphenylsulfone (PPS), ceramics, glass epoxy, metals, and resins filled with oxides or other fillers.

FIG. 5C is a sectional view schematically illustrating still another example of the dosing device 10C in accordance with another aspect of the present disclosure. The dosing device 10C is substantially the same as the dosing device 10B illustrated in FIG. 5B, except that the dosing device 10C further includes a pair of vibration transmission units 58A and 58B, each interposed between the ultrasonic oscillator 20 and an inner wall (e.g., opposing walls) of the housing 30. Specifically, the piezoelectric element forming the ultrasonic oscillator 20 is connected to the inner wall of the housing 30 via the first vibration transmission unit 58A on one side (e.g., in the Z axis direction) and via the second vibration transmission unit 58B on an opposite side (e.g., in the Z axis direction). In this configuration, the vibration of the piezoelectric element propagates directly to the housing 30 via both the first vibration transmission unit 58A and the second vibration transmission unit 58B, so that ultrasonic waves are efficiently radiated from the dosing device 10 in two directions. As compared with the single vibration transmission unit 58 of FIG. 5B, the pair of vibration transmission units 58A and 58B can further improve the sound pressure of the ultrasonic signal emitted from the dosing device 10 by providing a direct mechanical path from the piezoelectric element to the housing 30 on both sides of the ultrasonic oscillator 20. As with the vibration transmission unit 58 described with reference to FIG. 5B, each of the vibration transmission units 58A and 58B is preferably made of a material having a higher acoustic impedance than the sealant 54. Again, examples of materials that can be used for the vibration transmission units 58A and 58B include epoxy resin, polyphenylsulfone (PPS), ceramics, glass epoxy, metals, and resins filled with oxides or other fillers.

FIG. 5D is a sectional view schematically illustrating a further example of the dosing device in accordance with another aspect of the present disclosure. The dosing device 10D is substantially the same as the dosing device 10 illustrated in FIG. 5A, and includes the ultrasonic oscillator 20, the housing 30, the substrate 40, the power receiving coil 50, the battery 51, the biological information acquisition unit 52, and the sealant 54. However, in this configuration as the size of the housing 30 becomes smaller (e.g., in the Z axis direction), the piezoelectric element forming the ultrasonic oscillator 20 on the substrate 40 comes into direct contact with the inner wall of the housing 30. That is, the piezoelectric element itself is directly connected to the inner wall of the housing 30 without an intervening vibration transmission unit, such as that described above with respect to FIGS. 5B and 5C. In this configuration, the vibration of the piezoelectric element propagates directly to the housing 30 via the oscillator 20 and the components on which it is mounted, without requiring a separate vibration transmission member. As a result, ultrasonic waves are efficiently radiated from the dosing device 10, and the sound pressure of the ultrasonic signal emitted from the dosing device 10 can be improved. This configuration is particularly advantageous when the capsule size is reduced such that the internal dimensions of the housing 30 naturally bring the piezoelectric element (e.g., oscillator 20) into direct mechanical contact with the inner wall of the housing 30.

FIG. 5E is a sectional view schematically illustrating yet a further example of the dosing device in accordance with another aspect of the present disclosure. The dosing device 10E is substantially the same as the dosing device 10D illustrated in FIG. 5D, except the piezoelectric element is not in direct contact with the inner wall of the housing. Instead, as the size of the housing 30 becomes smaller, the battery 51 mounted on the substrate 40 comes into direct contact with the inner wall of the housing 30. That is, the battery 51 itself is directly connected to the inner wall of the housing 30, such that the vibration of the piezoelectric element of the oscillator 20 propagates through the substrate 40 and the battery 51 to the housing 30, so that ultrasonic waves are efficiently radiated from the dosing device 10. Because the battery 51 provides a direct mechanical path between the substrate 40 and the inner wall of the housing 30, the sound pressure of the ultrasonic signal emitted from the dosing device 10 can be improved as compared with a configuration in which only the sealant 54 surrounds the internal components. As with the configuration of FIG. 5D, this configuration is particularly advantageous when the capsule size is reduced such that the internal dimensions of the housing 30 naturally bring the battery 51 or other components mounted on the substrate 40 into direct mechanical contact with the inner wall of the housing 30.

FIG. 5F is a sectional view schematically illustrating another example of the dosing device in accordance with another aspect of the present disclosure. The dosing device 10F combines the configurations described above with respect to dosing device 10D and dosing device 10E. That is, as the size of the housing 30 becomes smaller, both the piezoelectric element forming the ultrasonic oscillator 20 and the battery 51 mounted on the substrate 40 come into direct contact with the inner wall of the housing 30. More specifically, the piezoelectric element is directly connected to the inner wall of the housing 30 (e.g., the upper wall in the Z axis direction) on one side of the substrate 40, and the battery 51 is directly connected to the inner wall of the housing 30 (e.g., the lower wall in the Z axis direction) on the opposite side of the substrate 40, without any intervening vibration transmission units. In this configuration, the vibration of the piezoelectric element 71 propagates directly to the housing 30 both via the piezoelectric element 71 itself and via the substrate 40 and the battery 51, providing two direct mechanical paths from the ultrasonic oscillator 20 to the housing 30. As a result, ultrasonic waves are efficiently radiated from the dosing device 10 in two directions in a similar configuration as described above. As with the configurations of FIGS. 5D and 5E, this configuration is particularly advantageous when the capsule size is reduced such that the internal dimensions of the housing 30 naturally bring both the piezoelectric element of oscillator 20 and the battery 51 into direct mechanical contact with the inner wall of the housing 30.

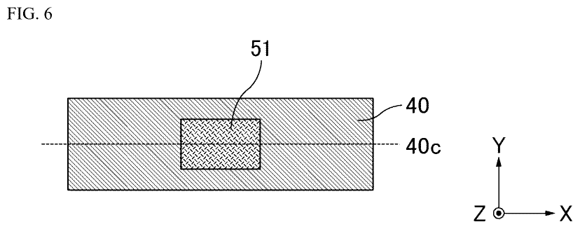

FIG. 6 is a plan view schematically illustrating an example of an arrangement relationship between a battery and a substrate on which the battery is mounted in the dosing device in accordance with an aspect of the present disclosure.

As illustrated in FIG. 6, the battery 51 is preferably disposed on a center line 40c in the longitudinal direction of the substrate 40 on which the battery 51 is mounted. That is, in plan view of the substrate 40, the battery 51 preferably overlaps with the center line 40c extending in the longitudinal direction of the substrate 40, that is, steps on the center line 40c. This makes it possible to avoid interference of a thick device, preferably the battery 51, with the capsule-shaped housing 30.

Note that although FIG. 6 illustrates the substrate 40 having a rectangular shape in plan view, the planar shape of the substrate 40 is not particularly limited as long as it fits in the dosing device 10, and may be a circular shape, an elliptical shape, or the like.

The biological information acquisition unit 52 acquires biological information such as a position in the body of the dosing device 10 taken, a temperature in the body, a pH of the stomach, and a vital sign such as intestinal activity. The temperature in the body may be a core body temperature. For example, the biological information acquisition unit 52 includes a timing unit for measuring time to measure an elapsed time after the dosing device 10 is taken into the body, and estimates the location of the dosing device 10 in the body according to the measured time. As another example, the biological information acquisition unit 52 includes sensors such as a temperature sensor, a pH sensor, and an acceleration sensor, and detects vital signs such as temperature in the body, pH of the stomach, and intestinal activity. The temperature sensor may include, for example, a thermistor. Note that, the biological information may include any piece of information related to a living body, and the biological information acquisition unit 52 can be configured to acquire any piece of biological information. The biological information acquisition unit 52 is controlled by the IC.

When the biological information is acquired by the biological information acquisition unit 52, the frequency of the ultrasonic wave emitted from the ultrasonic oscillator 20 may be changed for each type of biological information to be acquired.

Under the control of the IC, the A/D converter converts biological information that is analog information acquired by the biological information acquisition unit 52 into digital information, and outputs the biological information converted into digital information to the ultrasonic oscillator 20. Then, the biological information converted into digital information by the A/D converter is transmitted as an ultrasonic signal by the ultrasonic oscillator 20.

Power is supplied from the battery 51 to the ultrasonic oscillator 20, the IC, the biological information acquisition unit 52, and the A/D converter. Although FIG. 2 illustrates the configuration in which the biological information acquisition unit 52 is provided on the substrate 40 on which the ultrasonic oscillator 20 is provided, the place where the biological information acquisition unit 52, the IC, and the A/D converter are provided is not particularly limited to the substrate 40 on which the ultrasonic oscillator 20 is provided.

The sealant 54 is disposed inside the housing 30. The sealant 54 may fill substantially the entire space in the housing 30.

The sealant 54 has an acoustic impedance between an acoustic impedance of a material constituting the vibration surface of the ultrasonic oscillator 20 and an acoustic impedance of water, and is preferably filled between the vibration surface of the ultrasonic oscillator 20 and the outer surface 11 having the above-described curvature. In addition, it is preferable that the sealant 54 having such an acoustic impedance is filled between the vibration surface of the ultrasonic oscillator 20 and the first outer surface and/or the second outer surface having the above-described curvature. Here, since the acoustic impedance of water is substantially equal to the acoustic impedance of the living body, by filling the sealant 54 having the acoustic impedance, which falls between the acoustic impedance of the vibration surface of the ultrasonic oscillator 20 and the acoustic impedance of water, between the vibration surface of the ultrasonic oscillator 20 and the outer surface 11 having the curvature as described above, it is possible to effectively suppress the attenuation of the oscillating ultrasonic signal from the ultrasonic oscillator 20. As a result, the sound pressure of the ultrasonic signal emitted from the dosing device 10 into the body can be further improved.

More specifically, the acoustic impedance of PZT, which is a kind of material of the piezoelectric element, is 32 MPa·s/m³, and the acoustic impedance of water (≈living body) is 1.5 MPa·s/m³. Therefore, the acoustic impedance of the sealant 54 is preferably 1.5 MPa·s/m³ or more and 25 MPa·s/m³ or less.

As a material of such a sealant 54, for example, a resin filled with a filler such as an epoxy resin, polyphenylsulfone (PPS), a ceramic material, glass epoxy, a metal, or an oxide can be used.

Here, a result of simulating the maximum sound pressure of the ultrasonic signal emitted from the dosing device 10 by changing the material and the thickness of the sealant 54 will be described.

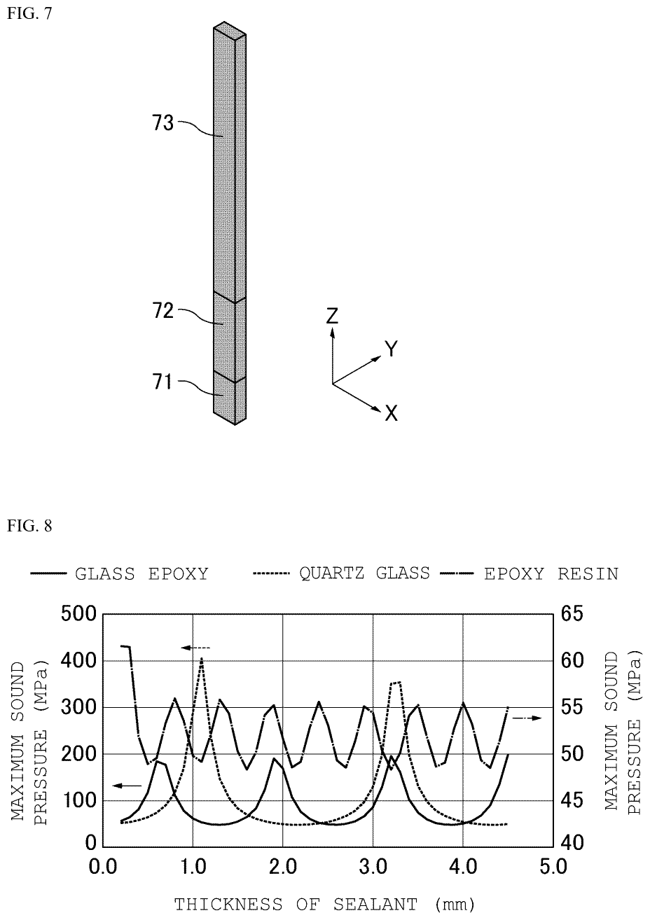

FIG. 7 is a diagram schematically illustrating an analysis model used to simulate the maximum sound pressure of an ultrasonic signal emitted from the dosing device.

In the analysis model illustrated in FIG. 7, a piezoelectric element 71, a sealant 72, and a living body 73 are arranged adjacent to each other in this order in the Z-axis direction. Then, using the analysis model illustrated in FIG. 7, resonance analysis has been performed in piezoelectric/acoustic wave analysis. The applied electric field is 75 V/mm, and the resonance frequency is 1.3 MHz. The material of the sealant 72 is glass epoxy, quartz glass, or epoxy resin. The thickness of the sealant 72 is in a range of 0.2 mm to 4.5 mm. The thickness of the sealant 72 is a dimension of the sealant 72 in the Z-axis direction. The acoustic impedance of the piezoelectric element 71 is 32 MPa·s/m³, the acoustic impedance of the living body 73 is 1.5 MPa s/m³, and the acoustic impedance of the sealant 72 is 6.2 MPa·s/m³ in the case of glass epoxy, 12.6 MPa·s/m³ in the case of quartz glass, and 1.7 MPa·s/m³ in the case of epoxy resin. An obtained result is shown in FIG. 8.

FIG. 8 is a graph showing the relationship between the maximum sound pressure of the ultrasonic signal and the thickness of the sealant, obtained from the simulation using the analysis model illustrated in FIG. 7. In FIG. 8, the left axis represents the maximum sound pressure in the case of glass epoxy and quartz glass, and the right axis represents the maximum sound pressure in the case of epoxy resin.

In addition, Table 1 below shows the maximum sound pressure obtained for each material of the sealant 72 and the thickness of the sealant 72 at that time.

TABLE 1

| | |
|---|---|
| Material | Maximum sound pressure |

| | | |
|---|---|---|
| Quartz glass | 405 MPa | @1.1 mm |
| Glass epoxy | 184 MPa | @0.6 mm |
| Epoxy resin | 62 MPa | @0.2 mm |

As illustrated in FIG. 8 and Table 1 above, it has been found that the maximum sound pressure can be effectively obtained by designing the thickness of the sealant 72 according to the material of the sealant 72.

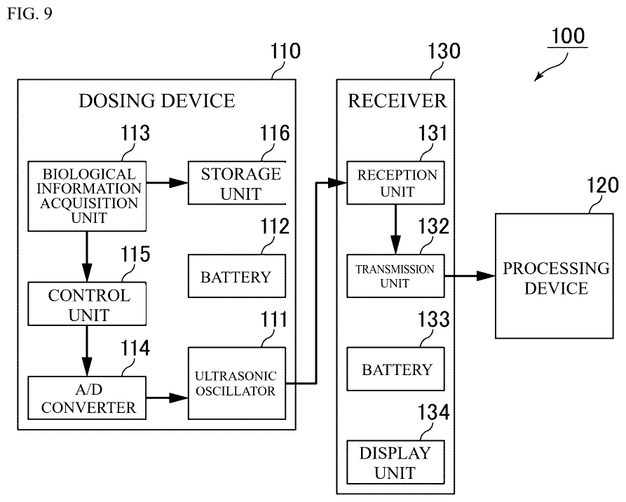

FIG. 9 is a block diagram schematically illustrating an example of an information acquisition system for the dosing device in accordance with another aspect of the present disclosure.

An information acquisition system 100 for a dosing device illustrated in FIG. 9 is a system for acquiring information from a dosing device 110, and includes the dosing device 110, a processing device 120, and a receiver 130.

The dosing device 110 includes, for example, an ultrasonic oscillator 111, a battery 112, a biological information acquisition unit 113, and an A/D converter 114, similarly to the dosing device 10 described above. In addition, the dosing device 110 includes a control unit 115 that performs control of timing of acquiring biological information, control of processing the acquired information and transmitting the processed information to the reception unit of the receiver 130, and the like.

The dosing device 110 may include a storage unit 116, and for example, the biological information acquired by the biological information acquisition unit 113 may be stored in the storage unit 116.

The receiver 130 acquires information from the dosing device 110 by receiving the oscillating ultrasonic signal from the ultrasonic oscillator 111 of the dosing device 110, and transmits the acquired information to the processing device 120. The receiver 130 is used by being attached to a body surface such as a torso, a neck, or a wrist of a recipient with a fixture such as a belt. The receiver 130 is mounted to be in direct contact with the body surface or to be in contact with the body surface via an inclusion such as a gel.

The receiver 130 includes, for example, a reception unit 131, a transmission unit 132, a battery 133, and a display unit 134.

The reception unit 131 receives the oscillating ultrasonic signal from the ultrasonic oscillator 111 of the dosing device 110. As a result, the receiver 130 acquires information from the dosing device 110, for example, biological information.

The reception unit 131 includes an ultrasonic receiver that receives an ultrasonic wave. A reception principle of the ultrasonic wave by the ultrasonic receiver is not particularly limited, but the ultrasonic receiver preferably includes a piezoelectric element, and the piezoelectric element preferably generates a voltage by receiving the ultrasonic wave. The piezoelectric element included in the reception unit 131 is also referred to as a piezoelectric vibrator. Materials that can be used for the piezoelectric element include lead zirconate titanate (PZT), barium titanate (BT), and potassium sodium niobate (KNN).

The transmission unit 132 transmits the information acquired from the dosing device 110 to the processing device 120 by wireless communication. The wireless communication is, for example, communication using a data communication line using a radio wave of a mobile phone, an Internet line, Bluetooth (registered trademark), or the like. Note that communication between the transmission unit 132 and the processing device 120 is not limited to wireless communication, and may be communication by wired connection such as USB cable connection, Ethernet cable connection, or serial cable connection.

The battery 133 supplies power to each unit of the receiver 130, and may be a chargeable/dischargeable secondary battery. A specific type of the battery 133 is not particularly limited.

The display unit 134 is configured to be able to display, for example, information acquired from the dosing device 110. Examples of the display unit 134 include a liquid crystal display and the like.

The processing device 120 acquires information transmitted from the transmission unit 132 of the receiver 130, that is, information from the dosing device 110. The processing device 120 performs various data processing and data management based on the acquired information. Specifically, for example, management/analysis of biological information acquired from the dosing device 110, management of medicine taken together with the dosing device 110, and the like are performed.

The information acquisition system 100 for the dosing device confirms whether the patient has taken the prescribed medicine by, for example, the following method.

The dosing device 110 is configured to be automatically turned on when taken out from the state of being enclosed in a container, and the dosing device 110 continues to emit an ultrasonic signal of a constant frequency from the ultrasonic oscillator 111 when turned on.

When the dosing device 110 is outside the body, air is interposed between the dosing device 110 and the receiver 130, so that the receiver 130 cannot receive the ultrasonic signal emitted from the dosing device 110. On the other hand, when the dosing device 110 is taken by the patient together with a medicine and the dosing device 110 enters the body, an ultrasonic signal can be transmitted with the acoustic impedance in the body, so that the receiver 130 can receive the ultrasonic signal emitted from the dosing device 110. That is, when the receiver 130 detects the ultrasonic signal, it is possible to detect that the medicine is taken together with the dosing device 110.

Then, when the receiver 130 detects that the medicine and the dosing device 110 have been taken, the receiver 130 transmits information indicating the fact to the processing device 120, and the presence or absence of dosage of the medicine, the dosage time, and the like are managed by the processing device 120 that has received the information.

In general, the description of the aspects disclosed should be considered as being illustrative in all respects and not being restrictive. The scope of the present disclosure is shown by the claims rather than by the above description and is intended to include meanings equivalent to the claims and all changes in the scope. While preferred aspects of the invention have been described above, it is to be understood that variations and modifications will be apparent to those skilled in the art without departing from the scope and spirit of the invention.

### DESCRIPTION OF REFERENCE SYMBOLS

10, 110: Dosing device
11: Outer surface
11a, 11c: First outer surface
11b, 11d: Second outer surface
20, 111: Ultrasonic oscillator
21: Vibration direction
21a, 21c: First direction
21b, 21d: Second direction
30: Housing
40: Substrate
40c: Center line in longitudinal direction of substrate
41: Mounting surface of substrate
50: Power receiving coil
51, 112, 133: Battery
52, 113: Biological information acquisition unit
54, 72: Sealant
71: Piezoelectric element
73: Living body
100: Information acquisition system
114: A/D converter
115: Control unit
116: Storage unit
120: Processing device
130: Receiver
131: Reception unit
132: Transmission unit
134: Display unit

1. A dosing device that emits information when the dosing device itself vibrates, the dosing device comprising:
an ultrasonic oscillator located within the dosing device,
wherein a curvature of an outer surface located on a vibration direction of the ultrasonic oscillator is 0.18 or more and 0.50 or less.

2. The dosing device according to claim 1, further comprising:
a sealant that has an acoustic impedance between an acoustic impedance of a material constituting a vibration surface of the ultrasonic oscillator and an acoustic impedance of water,
wherein the sealant is filled between the vibration surface of the ultrasonic oscillator and the outer surface.

3. The dosing device according to claim 1, further comprising:
a battery, and
a substrate on which the battery is mounted,
wherein the battery is disposed on a center line in a longitudinal direction of the substrate.

4. The dosing device according to claim 1, further comprising:
a substrate on which the ultrasonic oscillator is mounted,
wherein the vibration direction of the ultrasonic oscillator is a direction perpendicular to a mounting surface of the substrate.

5. The dosing device according to claim 4, wherein the outer surface is located on an opposite side of the substrate with respect to the ultrasonic oscillator.

6. The dosing device according to claim 1, further comprising:
a substrate on which the ultrasonic oscillator is mounted,
wherein the vibration direction of the ultrasonic oscillator is a direction parallel to a mounting surface of the substrate.

7. The dosing device according to claim 1, wherein the vibration direction of the ultrasonic oscillator includes a first direction and a second direction diametrically opposite to the first direction.

8. The dosing device according to claim 7, wherein the outer surface includes a first outer surface located at a place straight from the ultrasonic oscillator in the first direction and a second outer surface located at a place straight from the ultrasonic oscillator in the second direction.

9. The dosing device according to claim 8, wherein both curvatures of the first outer surface and the second outer surface are 0.18 or more and 0.50 or less.

10. The dosing device according to claim 1, wherein the ultrasonic oscillator includes a piezoelectric element.

11. The dosing device according to claim 10, further comprising at least one vibration transmission unit that connects the piezoelectric element to an inner wall of a housing of the dosing device, the at least one transmission unit being configured to propagate vibration of the piezoelectric element directly to the inner wall of the housing.

12. The dosing device according to claim 1, further comprising a capsule-shaped housing, and wherein the ultrasonic oscillator is disposed inside the capsule-shaped housing.

13. The dosing device according to claim 1, further comprising a biological information acquisition unit that acquires biological information.

14. The dosing device according to claim 13, further comprising an A/D converter that converts the biological information acquired by the biological information acquisition unit into digital information.

15. The dosing device according to claim 14, wherein the ultrasonic oscillator emits the biological information, which is converted into the digital information by the A/D converter, as an ultrasonic signal.

16. The dosing device according to claim 1, wherein the dosing device in an ingestible device.

17. The dosing device according to claim 3, wherein at least one of the ultrasonic oscillator and the battery is directly connected to an inner wall of the dosing device.

18. The dosing device according to claim 1, wherein the ultrasonic oscillator is located on a substrate.

19. An information acquisition system for a dosing device, comprising:
the dosing device that emits information when the dosing device itself vibrates, the dosing device comprising:
an ultrasonic oscillator that is provided inside,
wherein a curvature of an outer surface located on a vibration direction of the ultrasonic oscillator is 0.18 or more and 0.50 or less; and
the information acquisition system for the dosing device further comprises a processing device; and
a receiver that acquires the information from the dosing device by receiving an oscillating ultrasonic signal from the ultrasonic oscillator and transmits the acquired information to the processing device.

20. A wearable device configured to transmit information, comprising:
a rectangular prism-shaped piezoelectric element including opposing first and second main surfaces, opposing first and second side surfaces, and opposing third and fourth side surfaces,
a housing comprising the prism-shaped piezoelectric element,
wherein the first main surface of the prism-shaped piezoelectric element is fixed to an inner wall surface of the housing.
